(19) United States
(12) Patent Application Publication
(10) Pub. No.: US 2005/0043739 A1
(43) Pub. Date: Feb. 24, 2005

(54) Hybrid flexible drive shaft

(76) Inventors: Robert L. Sullivan, Warsaw, IN (US); Reese K. Myers, Warsaw, IN (US)

Correspondence Address: Todd T. Taylor;TAYLOR & AUST, P.C., P.O. Box 560, 142 S. Main St., Avilla, IN, 46710, US

(21) Appl. No.: 10/643,132

(22) Filed: Aug. 18, 2003

(51) Int. Cl. A61B 17/00; A61B 17/16; A61B 17/32

(52) U.S. Cl. 606/80; 606/79

(57) ABSTRACT

An orthopaedic reamer assembly, which includes a reamer and a flexible shaft connected to the reamer. The flexible shaft has both a longitudinal axis and a longitudinal length. The flexible shaft is comprised of a rigid material. The flexible shaft has a low ratio of an area moment of inertia about an axis perpendicular to the longitudinal axis versus the longitudinal length, the low ratio provides a flexibility in the flexible shaft.

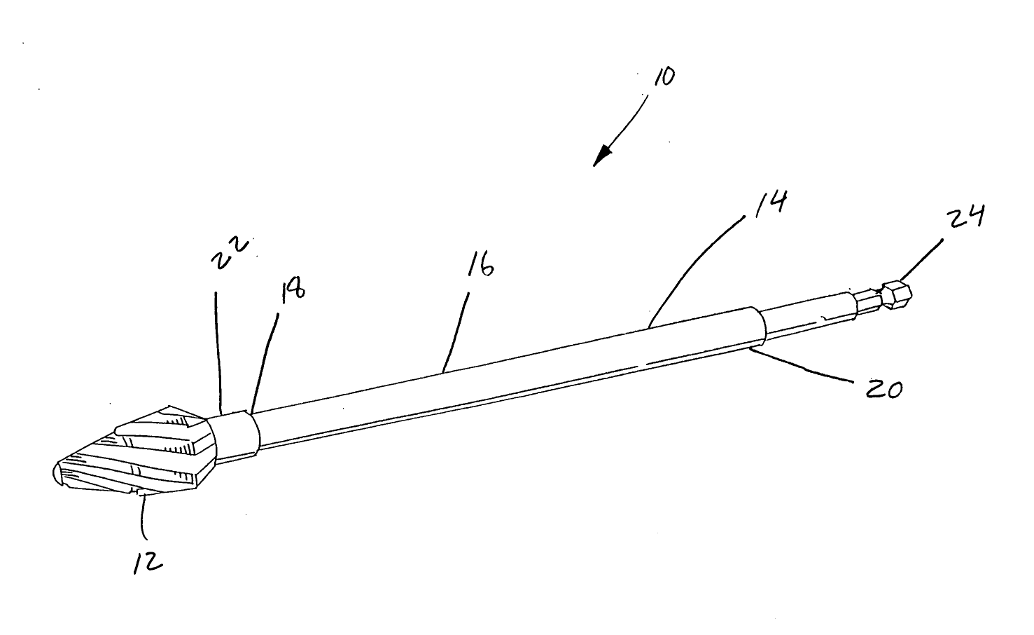

### BACKGROUND OF THE INVENTION

1. Field of the Invention

The present invention relates to orthopaedic reamers, and, more particularly, to flexible drive shafts used with orthopaedic reamers.

2. Description of the Related Art

Medullary canal reamers are used to enlarge the medullary canals of bone in preparation for the insertion of fixation devices, performing an intramedullary osteotomy, stimulating bone growth, the insertion of a plug to preclude bone cement from migrating while it is in its viscous state, and for other reasons. The medullary canals of bone are seldom straight. More typically the canal will have some degree of curvature to it. Should a straight and rigid series of reamers be employed to enlarge the canal there is considerable likelihood that the reamer, in not being capable of following the bones curvature, will not remove the desired uniform amount of bone tissue. In such a situation, excessive tissue removal occurring in at least one plane will be experienced as the reamer is advanced. For this reason medullary canals are almost always prepared with reamers having a flexible shaft.

Examples of known flexible medullary reamers include several types. One of the first to come into common usage consisted of spiral or helically wound metal wire(s) or strip(s), which composed the shaft of the reamer. A disadvantage of this design is that these reamers can be operated only in the forward mode of rotation. If operated in the reverse mode, which occasionally is required to free a lodged reamer and to facilitate normal removal, the shaft unwinds, thus rendering the reamer permanently deformed, unusable, and unrepairable. This adds considerably to the cost of maintaining a serviceable set of medullary reamers. Further, a lodged cutting head may subsequently be extremely difficult, if not impossible to remove without further violation of the involved bone and surrounding tissues. Another disadvantage of this design is the extreme difficulty in their proper and thorough cleaning after use. The spiral or helically wound metal shafts contain many voids of various sizes. Blood and tissue readily infiltrate such voids and become trapped within the confines of the shaft. When the reamer is in use the voids are considerably distorted and enlarged as the reamer is advanced towards and within the medullary canal, thus providing ready access for the particles of tissue. Prior to use, all medullary reamers are sterilized and hopefully, the blood and tissue particles not evacuated during the cleaning process and remaining within the interstices of the reamers, are at least rendered harmless. However, depending upon the amount and composition of the extraneous particles and their degree of isolation from the sterilizing process, the particles may not be rendered sterile. Even in a sterile condition these foreign particles may still cause problems of infection should they become dislodged from the confines of the reamer and come into contact with the patient's internal tissues. Medical professionals recognize this problem but acquiesce to using these reamers for lack of an acceptable alternative. A further disadvantage of this medullary reamer is that the torsional load it is subject to when in use results in poor power transfer and varying degrees of distortion of the shaft. If the power source providing the rotational energy to the reamer is great enough, the coils can tighten sufficiently to adversely affect the intended flexibility of the shaft. Another disadvantage associated with a spiral or helically wound reamer is the trauma it imposes to surrounding tissues. This results when the shaft of the reamer is not completely within the medullary canal as would occur during the initial reaming process. As the shaft rotates, that portion remaining outside of the medullary canal can become excessively flexed and distorted, thus enlarging the voids between the coils of the shaft. As the flexed shaft rotates, tissue lying outside of the canal and unintended for removal, becomes trapped within the voids and are torn from their underlying structures.

A second distinct type of flexible medullary reamer is described in the literature. The shaft of the reamer embodies a plurality of parallel, flexible elements joined together at opposite ends by means of a welded or soldered connection. A disadvantage of this shaft is that proper cleaning is difficult to accomplish. Towards its opposite ends where the individual elements converge, the elements come in close contact with each other until the final termination point is reached where they are permanently welded or soldered together to form a solid mass. Where these elements begin to converge, blood and tissue can readily become trapped and prove difficult to remove during the cleaning process. Another disadvantage of this flexible reamer is the excessive noise generated in its use which is caused by the individual elements being twisted and forcefully whipped into contact with each other. A further disadvantage of this type of flexible reamer occurs during usage. As rotation occurs, the individual elements spirally tighten around each other causing the shaft to become more rigid and thereby reducing the shaft's flexibility and increasing the likelihood of the attached cutting head not properly adhering to the central path of the medullary canal. Another disadvantage of this type of flexible reamer is the shaft's tendency, as it rotates but not yet fully within the confines of the medullary canal, to tear tissue from underlying structures as the individual elements are torsionally loaded and unloaded, thereby enlarging and contracting the spaces between the individual elements sufficiently to trap uninvolved tissue between the individual wires and tearing them free. Another disadvantage of this type of flexible reamer occurs in attempting to utilize the central bore of the reamer. The central bore is intended to receive a long small diameter guide pin which had previously been inserted into the medullary canal to act as a track for the advancing reamer. Except at its respective ends, this reamer lacks a well-defined and bordered central bore. Therefore, it is difficult to prevent the guide pin from exiting the reamer in the area of the free standing wires during the initial positioning of the guide pin within the reamer. A further disadvantage of this flexible shaft is the inefficient transfer of energy from the power source to the cutting head which is caused by the twisting and wrapping together of the individual elements as the reamer is rotated.

Nitinol tube shafts are used with flexible medullary reamers but have the disadvantages of being difficult to assemble and are very expensive.

What is needed in the art is a flexible driver which is easy to clean and sterilize, can be reversed without damaging the driver, is easy to manufacture and assemble and is reliable in service.

### SUMMARY OF THE INVENTION

The present invention provides a flexible driver with a flexible shaft of rigid material and used with orthopaedic reamers.

The present invention comprises, in one form thereof, an orthopaedic reamer assembly, which includes a reamer and a flexible shaft connected to the reamer. The flexible shaft has both a longitudinal axis and a longitudinal length. The flexible shaft is comprised of a rigid material. The flexible shaft has a low ratio of an area moment of inertia about an axis perpendicular to the longitudinal axis versus the longitudinal length, the low ratio provides a flexibility in the flexible shaft.

An advantage of the present invention is that it provides a flexible driver for orthopaedic reamers.

Another advantage is the present invention is easy to clean and sterilize.

Yet another advantage is the present invention can be reversed without damaging the driver.

A further advantage is the present invention is easy to manufacture and assemble.

A yet further advantage is the present invention provides reliable service.

### BRIEF DESCRIPTION OF THE DRAWINGS

The above-mentioned and other features and advantages of this invention, and the manner of attaining them, will become more apparent and the invention will be better understood by reference to the following description of an embodiment of the invention taken in conjunction with the accompanying drawing, which is a perspective view of an embodiment of a reamer assembly of the present invention. The exemplification set out herein illustrates one preferred embodiment of the invention, in one form, and such exemplification is not to be construed as limiting the scope of the invention in any manner.

### DETAILED DESCRIPTION OF THE INVENTION

Referring now to the sole drawing, there is shown a orthopaedic reamer assembly 10 which generally includes reamer 12 and driver 14.

Reamer 12 is shown as an intramedullary reamer; however, other type of orthopaedic reamers can be used.

Driver 14 generally includes a flexible shaft 16, with a reamer end or first end 18 and a chuck end or second end 20, and attachment element 22. Flexible shaft 16 can be comprised of material from the group consisting of polymers and composites thereof, and particularly, can be made of polyether ether ketone (PEEK) material. Polyether ether ketone is known for its inherent toughness, impact resistance, strength and outstanding chemical resistance. Further, polyether ether ketone is known as a rigid material making it a nonobvious choice for a flexible shaft. Flexible shaft 16 can have a circular cross-section perpendicular to a longitudinal direction of flexible shaft 16, however, other cross-sectional shapes such as elliptical, square, rectangular, polygonal, star shaped and other eclectic cross-sectional shapes are possible. Flexible shaft 16 can have a solid cross-section perpendicular to a longitudinal direction of flexible shaft 16, or alternatively, flexible shaft 16 can be of tubular form with a nonsolid cross-section perpendicular to a longitudinal direction of flexible shaft 16. A tubular flexible shaft 16 will therefore have a hollow longitudinal extent in the inner portion of flexible shaft 16.

Flexible shaft 16, although made of a rigid material such as PEEK, can be made flexible with a low ratio of an area moment of inertia about an axis perpendicular to the longitudinal axis of flexible shaft 16 versus the longitudinal length of flexible shaft 16, the low ratio such that there is a flexibility in the flexible shaft. Area moment of inertia is also known as the moment of inertia for an area or the second moment of an area about an axis. For example, for a circular cross-section of flexible shaft 16, the area moment of inertia about an axis perpendicular to the longitudinal axis and through the center of the cross-section is given by π(R⁴)4 where R is the radius of the cross-section. For a round tubular cross-section of flexible shaft 16, the area moment of inertia about an axis perpendicular to the longitudinal axis and through the center of the cross-section is given by π(R⁴−R₁⁴)/4 where R is the outer radius of the cross-section and R₁ is the inner radius of the cross-section. The flexibility or deflection in flexible shaft 16 will be a function of forces acting on the shaft such as the weights of reamer 12 and attachment element 22, the weight per unit length of flexible shaft 16, the force exerted on orthopaedic reamer assembly 10 during the reaming process, the modulus of elasticity of the material of flexible shaft 16, the longitudinal length of flexible shaft 16 and the appropriate area moment of inertia of flexible shaft 16. By choosing a suitably low ratio of an area moment of inertia about an axis perpendicular to the longitudinal axis of flexible shaft 16 versus the longitudinal length of flexible shaft 16, and giving consideration to the other factors affecting flexibility, flexible shaft 16 can be made flexible although made of a rigid material.

For example, the length of flexible shaft 16 between chuck end 20 and reamer end 18 can be 16 inches long. Flexible shaft 16 can be a circular tube with an outside diameter of 0.355 inches (R=0.1775 inches) and an inside diameter of 0.185 inches (R₁=0.0925). As stated above, the area moment of inertia about an axis perpendicular to the longitudinal axis and through the center of the cross-section is given by π(R⁴−R₁⁴)/4=π(0.1775⁴−0.0925⁴)/4=0.000722 inches⁴. For this example, the ratio of an area moment of inertia about an axis perpendicular to the longitudinal axis of flexible shaft 16 versus the longitudinal length of flexible shaft 16 is then 0.000722 inches⁴/16 inches=0.0000451 inches³. A suitable range of an area moment of inertia about an axis perpendicular to the longitudinal axis of flexible shaft 16 versus the longitudinal length of flexible shaft 16, for the present invention, can be approximately between 0.0003 inches³ to 0.000002 inches³, depending on the material chosen for flexible shaft 16, the size of the cross-section of flexible shaft 16, the degree of flexibility required and the longitudinal length of flexible shaft 16. The lower end of the range provides more flexibility. More preferably, a suitable range of an area moment of inertia about an axis perpendicular to the longitudinal axis of flexible shaft 16 versus the longitudinal length of flexible shaft 16, for the present invention, can be approximately between 0.0002 inches³to 0.00001 inches³.

Attachment element 22 is connected at one end to reamer end 18 of flexible shaft 16 and can be releasably connected to reamer 12. Attachment element 12 can be comprised of a metal material. Alternatively, attachment element 12 can be comprised of a material from the group consisting of polymers and composites thereof, and particularly, can be made of polyether ether ketone material.

Drive connection 24 is connected to flexible shaft 16 at chuck end 20. Drive connection 24 can be comprised of a metal material. Alternatively, drive connection 24 can be comprised of a material from the group consisting of polymers and composites thereof, and particularly, can be made of polyether ether ketone material.

Both attachment element 22 and drive connection 24 can be attached to flexible shaft 16 using a thermal assembly method, epoxy or fastening elements.

Alternatively, driver 14, including drive connection 24 and attachment element 22, can be a monolithic structure comprised of a material from the group consisting of polymers and composites thereof, and particularly, can be made of polyether ether ketone material.

In use, driver 14 is assembled by connecting flexible shaft 16 to attachment element 22 and drive connection 24. Flexible shaft 16, although manufactured from a known rigid material such as PEEK, can be made flexible, for example, by making the area moment of inertia of a cross-section perpendicular to the longitudinal direction of flexible shaft 16 relatively small when compared to the longitudinal length of flexible shaft 16. Before a reaming operation, driver 14 is releasably connected to reamer 12 at attachment element 22. Drive connection 24 is then connected to a rotary tool (not shown) to perform a orthopaedic reaming function such as an intramedullary osteotomy or other operations.

While this invention has been described as having a preferred design, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

1. An orthopaedic reamer assembly, comprising:
a reamer;
a flexible shaft connected to said reamer, said flexible shaft having both a longitudinal axis and a longitudinal length, said flexible shaft being comprised of a rigid material, said flexible shaft having a low ratio of an area moment of inertia about an axis perpendicular to said longitudinal axis versus said longitudinal length, said low ratio providing a flexibility in said flexible shaft.

2. The orthopaedic reamer assembly of claim 1, wherein said rigid material is comprised of polyether ether ketone.

3. The orthopaedic reamer assembly of claim 1, wherein said low ratio of an area moment of inertia about an axis perpendicular to said longitudinal axis versus said longitudinal length is approximately between 0.0003 inches³ to 0.000002 inches³.

4. The orthopaedic reamer assembly of claim 1, wherein said low ratio of an area moment of inertia about an axis perpendicular to said longitudinal axis versus said longitudinal length is approximately between 0.0002 inches³ to 0.00001 inches³.

5. The orthopaedic reamer assembly of claim 1, further including both a reamer end on said flexible shaft and an attachment element connected to said reamer end, said attachment element releasably connected to said reamer, said attachment element being comprised of a metal material, said rigid material being comprised of a material from the group consisting of polymers and composites thereof.

6. The orthopaedic reamer assembly of claim 5, wherein said rigid material is comprised of polyether ether ketone.

7. The orthopaedic reamer assembly of claim 5, wherein said flexible shaft has a chuck end, said chuck end being made of metal.

8. The orthopaedic reamer assembly of claim 1, wherein said flexible shaft has a solid cross-section along said longitudinal length.

9. The orthopaedic reamer assembly of claim 1, wherein said flexible shaft has a tubular form.

10. The orthopaedic reamer assembly of claim 1, wherein said flexible shaft is fixedly attached to said reamer.

11. An orthopaedic driver for rotatably driving a reamer, comprising:
a flexible shaft configured for connection to the reamer, said flexible shaft having both a longitudinal axis and a longitudinal length, said flexible shaft being comprised of a rigid material, said flexible shaft having a low ratio of an area moment of inertia about an axis perpendicular to said longitudinal axis versus said longitudinal length, said low ratio providing a flexibility in said flexible shaft.

12. The orthopaedic reamer assembly of claim 11, wherein said rigid material is comprised of polyether ether ketone.

13. The orthopaedic reamer assembly of claim 11, wherein said low ratio of an area moment of inertia about an axis perpendicular to said longitudinal axis versus said longitudinal length is approximately between 0.0003 inches³ to 0.000002 inches³.

14. The orthopaedic reamer assembly of claim 11, wherein said low ratio of an area moment of inertia about an axis perpendicular to said longitudinal axis versus said longitudinal length is approximately between 0.0002 inches³ to 0.00001 inches³.

15. The orthopaedic reamer assembly of claim 11, further including both a reamer end on said flexible shaft and an attachment element connected to said reamer end, said attachment element configured for releasable connection to the reamer, said attachment element being comprised of a metal material, said rigid material being comprised of a material from the group consisting of polymers and composites thereof.

16. The orthopaedic reamer assembly of claim 15, wherein said rigid material is comprised of polyether ether ketone.

17. The orthopaedic reamer assembly of claim 15, wherein said flexible shaft has a chuck end, said chuck end being made of metal.

18. The orthopaedic reamer assembly of claim 11, wherein said flexible shaft has a solid cross-section along said longitudinal length.

19. The orthopaedic reamer assembly of claim 11, wherein said flexible shaft has a tubular form.

20. A method of manufacturing an othopaedic reamer, comprising the steps of:
assembling a flexible shaft, said flexible shaft having both a longitudinal axis and a longitudinal length, said flexible shaft being comprised of a rigid material, said flexible shaft having a low ratio of an area moment of inertia about an axis perpendicular to said longitudinal axis versus said longitudinal length, said low ratio providing a flexibility in said flexible shaft;
connecting said flexible shaft to a reamer.

21. The method of claim 20, wherein said rigid material is comprised of polyether ether ketone.

22. The method of claim 20, wherein said flexible shaft is releasably connected to said reamer.

23. The orthopaedic reamer assembly of claim 20, wherein said low ratio of an area moment of inertia about an axis perpendicular to said longitudinal axis versus said longitudinal length is approximately between 0.0003 inches³ to 0.000002 inches³.

24. The orthopaedic reamer assembly of claim 20, wherein said low ratio of an area moment of inertia about an axis perpendicular to said longitudinal axis versus said longitudinal length is approximately between 0.0002 inches³ to 0.00001 inches³.
